(19) United States
(12) Patent Application Publication
(10) Pub. No.: US 2023/0011767 A1
(43) Pub. Date: Jan. 12, 2023

(54) DIRECT ORAL ANTICOAGULANT-ELUTING MEDICAL DEVICE

(71) Applicant: BOSTON SCIENTIFIC SCIMED, INC., Maple Grove, MN (US)

(72) Inventors: Steven L. Kangas, Woodbury, MN (US); Edward Kopesky, Mahtomedi, MN (US); Yen-Lane Chen, New Brighton, MN (US)

(73) Assignee: BOSTON SCIENTIFIC SCIMED, INC., MAPLE GROVE, MN (US)

(21) Appl. No.: 17/860,848

(22) Filed: Jul. 8, 2022

Related U.S. Application Data
(60) Provisional application No. 63/220,013, filed on Jul. 9, 2021.

(51) Int. Cl. A61B 17/12; A61K 31/4545; A61K 31/5377; A61K 31/444

(52) U.S. Cl. CPC A61B 17/12177; A61B 17/12122; A61B 17/12172; A61K 31/4545; A61K 31/5377; A61K 31/444; A61B 2017/00597

(57) ABSTRACT

A device for permanent placement across an atrial appendage ostium in a patient includes a support structure having a contracted delivery configuration and an expanded deployed configuration defining a radially enlarged portion to permanently engage an interior wall of the atrial appendage, a membrane attached to the support structure and configured to extend across the ostium of the atrial appendage when the support structure is in the expanded deployed configuration, and a polymer coating disposed on at least one of the support structure and the membrane, the polymer coating including a direct oral anticoagulant (DOAC) dispersed in a polymer.

### CROSS-REFERENCE TO RELATED APPLICATIONS

This application claims the benefit of priority of U.S. Provisional Application No. 63/220,013 filed Jul. 9, 2021, the entire disclosure of which is hereby incorporated by reference.

### TECHNICAL FIELD

The disclosure pertains to medical devices and more particularly to anticoagulant coatings on medical devices for preventing device related thrombosis, and methods for using such medical devices.

### BACKGROUND

A wide variety of medical devices have been developed for medical use including, for example, medical devices utilized to treat non-valvular atrial fibrillation. These medical devices may be used to isolate the left atrial appendage (LAA). Implanted medical devices are available for insertion into the LAA to block blood clots from passing out of the heart into the systemic circulation. Over time, the exposed surface structures of the implanted medical device spanning the ostium of the LAA becomes covered with tissue. Of the known medical devices and methods, each has certain advantages and disadvantages. There is an ongoing need to provide alternative medical devices as well as alternative methods for manufacturing and using the medical devices.

### SUMMARY

This disclosure provides design, material, manufacturing method, and use alternatives for medical devices. An example medical device includes a device for permanent placement across an atrial appendage ostium in a patient, comprising a support structure having a contracted delivery configuration and an expanded deployed configuration defining a radially enlarged portion to permanently engage an interior wall of the atrial appendage, a membrane attached to the support structure and configured to extend across the ostium of the atrial appendage when the support structure is in the expanded deployed configuration, and a polymer coating disposed on at least one of the support structure and the membrane, the polymer coating including a direct oral anticoagulant (DOAC) dispersed in a polymer.

Alternatively or additionally to the embodiment above, the DOAC is apixaban, rivaroxaban, or edoxaban.

Alternatively or additionally to any of the embodiments above, the polymer coating is disposed on the membrane.

Alternatively or additionally to any of the embodiments above, the DOAC is present in the polymer coating in a ratio of 60/40 to 90/10 weight/weight of polymer to DOAC.

Alternatively or additionally to any of the embodiments above, the DOAC is present in the polymer coating in an amount of between 10-10,000 μg.

Alternatively or additionally to any of the embodiments above, the polymer coating includes the DOAC in a coat density of 100-50,000 ng DOAC/mm² of membrane surface area.

Alternatively or additionally to any of the embodiments above, the polymer is poly(vinylidene fluoride)-co-hexafluoropropylene and the polymer coating has a thickness of about 10-20 μm.

Alternatively or additionally to any of the embodiments above, the polymer coating is disposed directly on the support structure.

Alternatively or additionally to any of the embodiments above, the polymer coating has a thickness of 20 μm.

Alternatively or additionally to any of the embodiments above, the DOAC is present in an amount of 100-300 μg.

Alternatively or additionally to any of the embodiments above, the polymer coating is disposed on a proximal end of the support structure.

Alternatively or additionally to any of the embodiments above, the polymer coating is a 1-10 μm thick film laminated to the membrane.

Alternatively or additionally to any of the embodiments above, the film contains 100-450 μg of the DOAC.

Alternatively or additionally to any of the embodiments above, the film includes a plurality of pores.

Alternatively or additionally to any of the embodiments above, the plurality of pores is 20-150 μm.

Alternatively or additionally to any of the embodiments above, the film is disposed on an atrial face of the membrane.

Alternatively or additionally to any of the embodiments above, the film includes a base layer with the DOAC and a top layer with a modulating compound.

Another example device for permanent placement across a left atrial appendage ostium in a patient comprises a self-expanding support structure having a first contracted shape for delivery and a second expanded shape configured to engage an interior wall of the left atrial appendage, the support structure including a plurality of struts defining an atrial face extending across the left atrial appendage ostium when in the second expanded shape, a membrane disposed on the atrial face and extending along at least a portion of a side surface of the support structure and configured to extend across the atrial appendage ostium in the second expanded shape, and a polymer drug coating disposed on one or both of the support structure and the membrane, the polymer drug coating including a direct oral anticoagulant (DOAC) dispersed in a polymer.

Alternatively or additionally the embodiment above, the polymer drug coating is a 1-10 μm thick film laminated directly onto the membrane.

An example method of making an expandable device for permanent placement across a left atrial appendage ostium in a patient comprises forming an expandable support structure having a contracted delivery configuration and an expanded deployed configuration defining a radially enlarged portion sized to permanently engage an interior wall of the left atrial appendage, attaching a membrane over at least a proximal end of the support structure, and applying a polymer coating containing a direct oral anticoagulant dispersed in a polymer to at least one of the support structure and the membrane.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 illustrates a portion of an example medical device according to the present disclosure;

FIG. 2 illustrates the medial device shown in FIG. 1 with a membrane;

FIG. 3 is the example medical device shown in FIG. 2 deployed within a partial cross-sectional view of the left atrial appendage of a patient;

FIG. 4A is a partial cross-sectional view of an example medical device with a polymer thin film disposed over the membrane;

FIG. 4B is a partial cross-sectional view of another example medical device with a polymer thin film disposed between the membrane and the support structure;

FIG. 5 illustrates blood clots formed on membranes coated with a polymer and various direct anticoagulants at various time points;

FIG. 6 is a graph showing the clot weights from FIG. 5;

FIG. 7 is a top view of a laser cut 25 μm thick polyethylene terephthalate (PET) film;

FIG. 8 is a graph showing drug release over time from spray coated PET films;

FIGS. 9A-9D show control and spray-coated devices after blood exposure; and

FIGS. 10A and 10B show a partially masked spray-coated device before and after blood exposure.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

Non-valvular atrial fibrillation (a-fib) is a condition that puts a patient at high risk for stroke due to diminished blood flow through the left atrial appendage (LAA), resulting in low blood flow conditions favorable for clot formation. As a result of this condition, patients with a-fib may require oral anticoagulated therapy for life. Oral anticoagulants are a systemic treatment and may present unique risks for the patient, especially those with high risk for bleeding. In an effort to reduce the occurrence of thrombi formation within the LAA and prevent thrombi from entering the blood stream from within the LAA, medical devices have been developed that close off the LAA from the heart and/or circulatory system, thereby lowering the risk of stroke and other embolic ischemic events due to thrombolytic material entering the blood stream from the left atrial appendage.

It is often recommended that patients receiving an LAA occlusion device take oral anticoagulants (OACs) for about 45 days following implantation, followed by at least six months of dual antiplatelet therapy (DAPT). OAC therapy after implantation of an LAA occlusion device is to ensure low risk of device related thrombosis (DRT) in the first six weeks post implant while tissue grows over the device. OAC therapy is highly effective at reducing risk of DRT but because it is a systemic treatment, it may have serious systemic negative effects in some patients such as brain bleed, gastro-intestinal bleeding, and internal bleeding as a result of blunt force trauma, such as falls. For patients at high risk for bleeding, it would be desirable to avoid taking OACs after implantation of the LAA occlusion device. There may also be an issue of noncompliance on the part of patients in taking OACs after implantation of an LAA occlusion device. About 3-4% of patients develop a device related thrombus after cessation of OAC, between 45 days and 1 year post implantation. Patients that develop a DRT must go back on OAC until the DRT resolves.

Applicants have developed an occlusion device that (a) eliminates the need for systemic OAC therapy after the device implantation and (b) continues to reduce the risk of DRT in the long term. The occlusion device takes the conventional systemic OAC therapy and instead uses direct oral anticoagulants (DOACs) and provides them only on the surface of the device where they are needed, thus providing the advantage of reducing the potential adverse effects of systemic OAC therapy. This may be accomplished by incorporating DOACs into a polymer coating disposed on one or more portions of an LAA occlusion device. The occlusion device may include a support structure and a membrane, with the polymer coating disposed on one or both of the support structure and the membrane.

Unlike conventional anticoagulants such as heparin and warfarin which inhibit various cofactors in the clotting cascade, and which may contribute to the serious systemic negative effects, the category of anticoagulants known as direct oral anticoagulants (DOACs) bind directly to specific clotting factors. Examples of DOACs include apixaban, rivaroxaban, edoxaban, dabigatran, betrixaban, and argatroban, which directly bind to factor Xa, and dabigatran, which directly binds to factor IIa. The LAA occlusion device provides a way of achieving localized release of these DOAC's at the surface of the device.

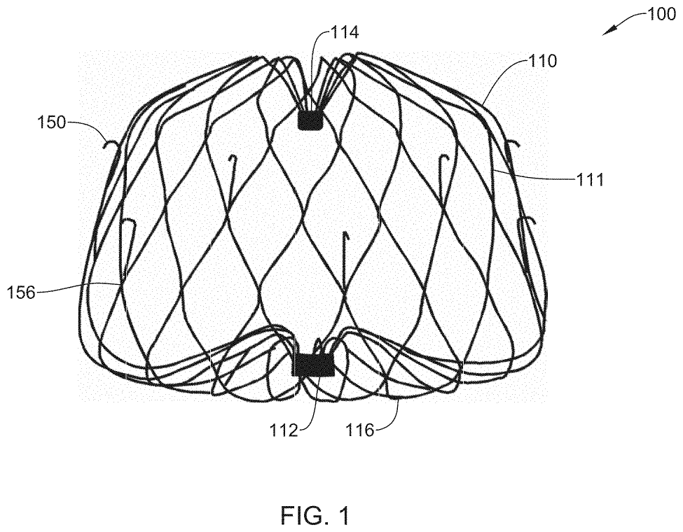

FIG. 1 illustrates a perspective view of a portion of an example LAA occlusion device or implant 100. The implant 100 may include a self-expanding support structure 110 extending from a proximal collar 112 to a distal collar 114. In some embodiments, the support structure 110 may include a plurality of struts 111 forming a lattice. The support structure 110 including the proximal collar 112 and struts 111 may be monolithic or it may be formed of multiple parts. The proximal end 116 of the support structure 110 will face the left atrium when implanted in the LAA, and may be referred to as the atrial face of the support structure. In some embodiments, the proximal and distal end portions of the struts may be attached directly to the proximal and/or distal collar(s), respectively. In some embodiments, the support structure 110 may include a plurality of anchors 150 provided to secure the implant 100 to the lateral wall of the left atrial appendage after deployment and thereby inhibit proximal movement of the implant 100 relative to the LAA. In the illustrated embodiment, each of the plurality of anchors 150 extend distally from a strut node junction 156. However, it will be understood that other alternate positions and arrangements of the plurality of anchors 150 are also possible. The support structure 110 has a contracted delivery shape or configuration and an expanded deployed shape or configuration, as shown in FIG. 1, in which the support structure 110 defines a radially enlarged portion to permanently engage an interior wall of the atrial appendage. When the support structure 110 is in the expanded configuration the atrial face is configured to extend completely across the LAA ostium.

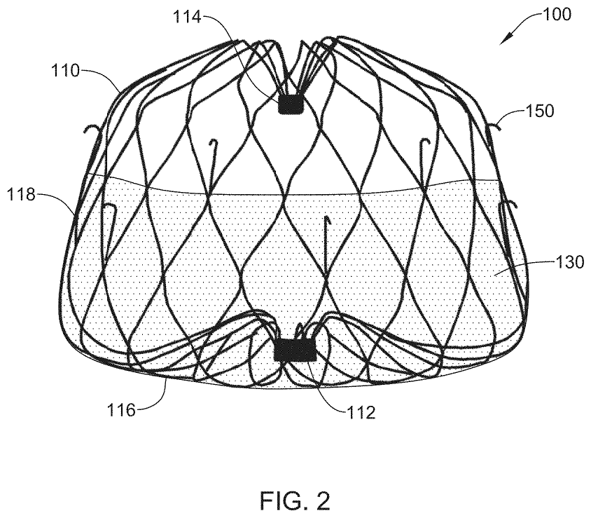

FIG. 2 illustrates the example implant 100 shown in FIG. 1 with a membrane 130 disposed over at least a portion of the support structure 110. In some embodiments, at least some of the plurality of anchors 150 project through the membrane 130. In some embodiments, the membrane 130 may be attached to the support structure 110 at each anchor 150, for example, by passing each anchor 150 through the membrane 130, such as through a pore or aperture. In other embodiments, the membrane 130 may be attached to the support structure 110 by other suitable attachment means, such as but not limited to, adhesive(s), sutures or thread(s), welding or soldering, or combinations thereof. In some embodiments, the membrane 130 may be permeable or impermeable to blood and/or other fluids, such as water. In some embodiments, the membrane 130 may include a polymeric membrane, a metallic or polymeric mesh, a porous filter-like material, or other suitable construction. The membrane 130 may extend completely over the proximal end 116 or atrial face of the support structure 110. In some examples, the membrane 130 may also extend along at least a portion of the side surface 118 of the support structure 110, as illustrated in FIG. 2. In this manner, the membrane 130 is configured to extend across the ostium of the LAA when the support structure 110 is in the expanded deployed configuration. In some embodiments, the membrane 130 prevents thrombi (i.e. blood clots, etc.) that may have formed in the LAA from passing through the membrane 130 and out of the LAA into the blood stream. In some embodiments, the membrane 130 promotes endothelization after implantation, thereby effectively removing the LAA from the patient's circulatory system.

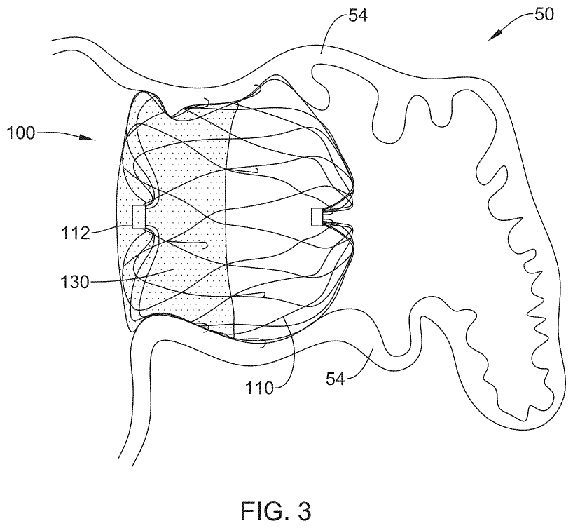

FIG. 3 illustrates a partial cross-sectional view of the implant 100 disposed within an example left atrial appendage 50, in a deployed position. As can be seen in FIG. 3, the support structure 110 may be compliant and substantially conform to and/or be in sealing engagement with the shape and/or geometry of the lateral wall 54 of the left atrial appendage 50 in the deployed position. At its largest size, extent, or shape, the implant 100 may expand to a fully unconstrained position in the deployed position.

The above described LAA occlusion implant 100 is just one of many different LAA implants that may incorporate the DOAC-containing polymer coating. The following examples refer to an LAA occlusion device such as those described in U.S. Pat. Nos. 6,652,556, 6,689,150, 6,949,113, 7,727,189, 9,913,652, and 11,241,237, the disclosures of which are incorporated herein by reference.

The polymer coating may include a hemocompatible polymer such as poly(vinylidene fluoride)-co-hexafluoropropylene (PVDF-HFP), and an anticoagulant such as a direct oral anticoagulant (DOAC). The resulting drug coating may be applied to the membrane 130 and/or the support structure 110 to act as a drug depot for sustained localized release. The relatively large size of the implant provides the ability to build in drug reservoirs to provide long duration (1 year) release of DOAC and other drugs to locally treat DRT and other cardiac disease states.

In one embodiment, a polymer coating such as PVDF-HFP, and one or more DOACs are dissolved in a solvent suitable for dissolving the polymer. This solution may be applied directly to the membrane 130 by a dip coating or spray process. Spray coating may result in the polymer coating being disposed only on one side of the membrane 130, with the uncoated side being attached to the support structure 110. The spray coating may be performed on the membrane before the membrane is attached to the support structure, or it may be performed after the membrane is attached to the support structure. The polymer coating may be applied to achieve a coat density of between 100-50,000 ng drug/mm² on the membrane. In some examples, the polymer coating may be applied to achieve a coat density of 10,000 ng drug/mm² on the membrane. The polymer to drug ratio may be, for example, 50/50 to 90/10 (weight/weight). Some examples include a polymer to drug ration of 60/40, 70/30, or 80/20 (weight/weight). The average coating thickness on the membrane may be about 10-30 μm. In one example, the amount of drug contained in the coated membrane of a 24 mm device may about 10-20,000 μg.

In another example, the drug-containing polymer coating may be disposed directly onto the support structure 110. The support structure 110 may be nitinol. In some examples, the proximal end 116 only of the support structure 110 may be conformally coated with a mixture of PVDF-HFP and DOAC. In other examples, the polymer coating may be disposed on the proximal end 116 and at least a portion of the side surface 118 of the support structure 110. Alternatively, the entire support structure 110 may be coated with the polymer coating. A significantly thicker coating may be achievable when the polymer coating is disposed directly on the support structure 110 compared to coating the membrane 130. The membrane coating thickness is limited due to the impact of folding and unfolding of the membrane during loading and deployment of the device. A thicker membrane coating may crack and flake or impede the folding and unfolding process. Polymer coatings up to 30 μm thickness and anticoagulant content of 100-300 μg are achievable by direct coating of the support structure 110. The significantly thicker coatings on the support structure 110 may lead to a longer drug release time compared to coating the membrane 130. In some examples, a slower drug release may be achieved by applying a basecoat containing the drug, drying the device and then applying a topcoat without the drug. In other examples, the drug coating may be applied to a select area or region of the device by masking the remaining device during the coating process. This may allow for the drug to be applied only on a specified desired region.

In some examples, both the membrane 130 and the support structure 110 may be coated with the polymer and DOAC coating. This may provide an increased amount of drug to be delivered as compared to coating only the support structure or the membrane alone.

In a further example, a thin layer of PVDF-HFP with DOAC is formed into a thin film 160 and laminated directly onto the membrane 130.

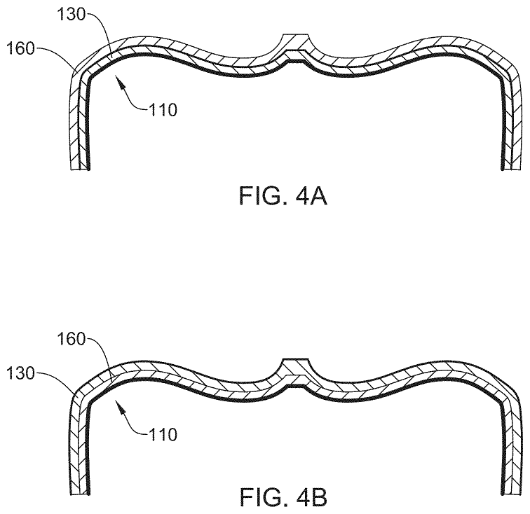

The film may be very thin relative to the membrane 130, which may be polyethylene terephthalate (PET). For example, the thin film 160 of polymer and DOAC may have a thickness of 1-10 μm compared to a 120 μm thick membrane 130. The polymer film is very flexible and compliant relative to the membrane 130 and does not negatively impact loading or deployment of the device. The thin film 160 may be significantly thicker, such as 10-100 times thicker, than a dip or spray coating of polymer and DOAC directly on the membrane 130. In some examples, the film may be formed of multiple layers. The thicker film or multi-layer film may provide for a longer duration for drug release because the film can hold significantly more drug. For example, the thin film may contain about 100-450 μs of anticoagulant drug. In some examples, the thin film 160 may be disposed on the atrial face of the membrane 130, as shown in FIG. 4A. In other examples, the thin film 160 may be sandwiched between the membrane 130 and the support structure 110, as shown in FIG. 4B.

The thin film 160 may also be made porous to enable blood flow through the device acutely. The pores may be formed during the film-making process or after forming the film such as by laser cutting or other processes such as high temperature annealing. The pores may be about 20 μm to 150 μm in diameter, to match the pores in the membrane 130.

In some examples, the thin film 160 may include multiple layers that may be different. For example, the thin film 160 may include a base layer of polymer and anticoagulant and a top layer including a modulating compound.

In some examples, a method of manufacturing the implant 100 may include the steps of (1) forming an expandable support structure 110 having a contracted delivery configuration and an expanded deployed configuration defining a radially enlarged portion sized to permanently engage the interior wall of the left atrial appendage; (2) attaching a membrane 130 over at least the proximal end 116 of the support structure 110; and (3) applying a polymer coating containing a DOAC dispersed in a polymer to at least one of the support structure 110 and the membrane 130. The polymer coating may be applied directly to the membrane 130, directly to the support structure 110, both the membrane 130 and the support structure 110, or the polymer coating may be formed into a thin film 160 that is then laminated to the membrane 130.

In some examples, the method of forming the expandable support structure may include the steps of (a) obtaining an elongate tubular member having a lumen extending therethrough and an annular ring member; (b) laser cutting the tubular member to form a proximal collar 112, a plurality of struts 111 having free distal ends, and a plurality of anchors 150 interspersed among the plurality of struts, as a single monolithic structure; (c) forming the plurality of struts 111 into a lattice of generally diamond-shaped wire portions; and (d) fixedly attaching the plurality of free distal ends of the struts 111 to the distal collar 114. The step of attaching the membrane 130 over at least the proximal end 116 of the support structure 110 may include attaching the membrane 130 over the proximal end 116 and along at least a portion of the side surface 118 such that the plurality of anchors 150 extends through the membrane 130.

### Example 1: Coating Disposed Directly on Membrane

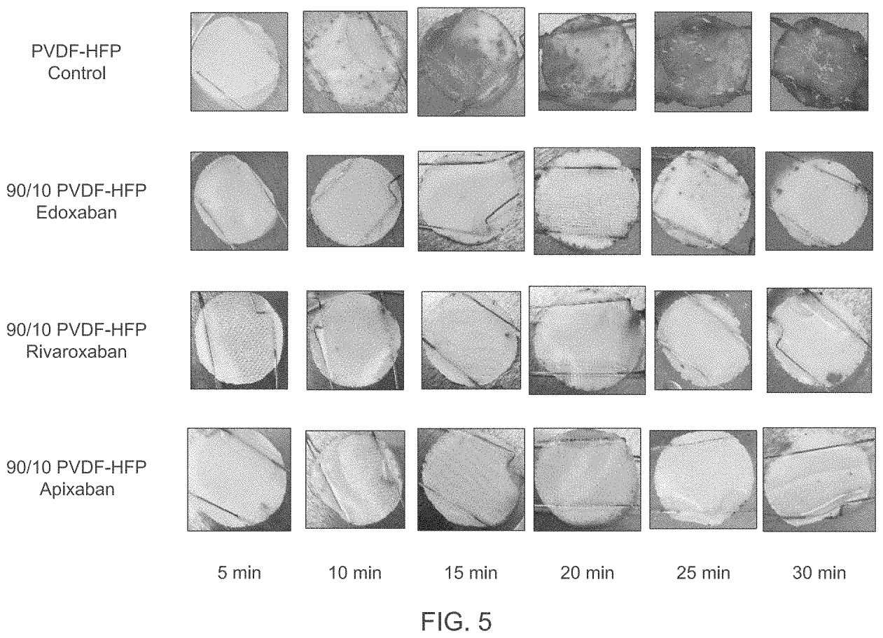

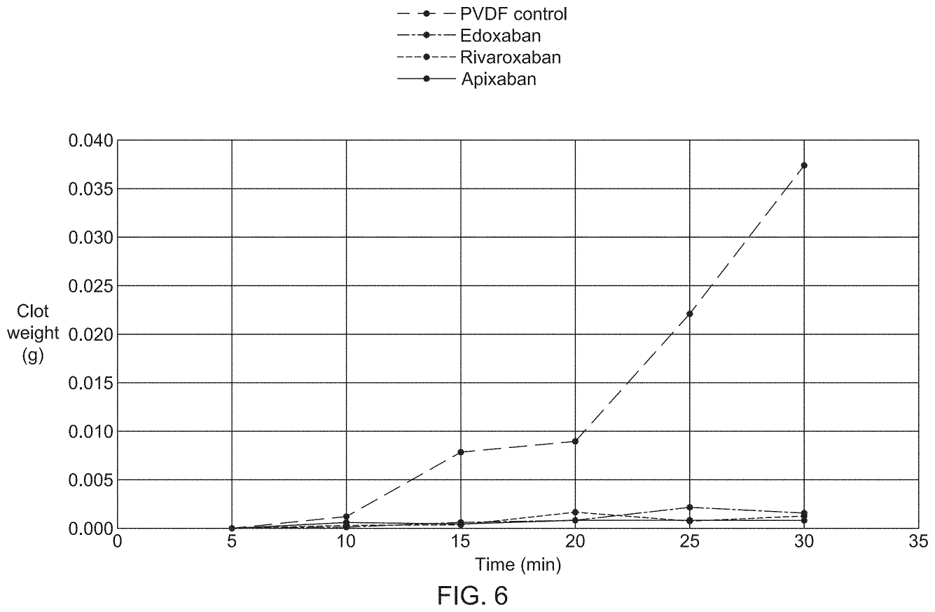

A solution of PVDF-HFP and DOAC was prepared in 80/20 acetone/DMSO (wt/wt). The PVDF-HFP to drug ratio was 90/10 (wt/wt) and the solution solids was 0.7%. The DOAC drugs evaluated were apixaban (Eliquis®), rivaroxaban (Xarelto®) and edoxaban (Savaysa®). 15 mm diameter polyethylene terephthalate (PET) fabric disks were dip coated into the polymer/drug solution at a dip speed of 5 mm/sec. The coated disks were dried for 30 min at 125° C. in a convection oven. The drug coated disks and PVDF-HFP only control disks were placed in cups containing heparinized bovine blood adjusted to an active clotting time (ACT) of about 190 seconds using protamine. The cups were placed on an orbital shaker incubator at 37° C. and disks were removed at various time points and imaged. Images are shown in FIG. 5. The disks were then dried and weighed to determine clot weight. Clot weights are shown in FIG. 6. As seen in FIG. 5, all three drugs showed significantly less thrombus (clots) compared to the PVDF-HFP coated control, showing the drugs are highly effective at preventing acute thrombus from forming on the fabric. The clot weights provided in FIG. 6 verify the minimal amount of thrombus formed on the DOAC treated fabric.

### Example 2: Coating Disposed on Laser Cut PET Film

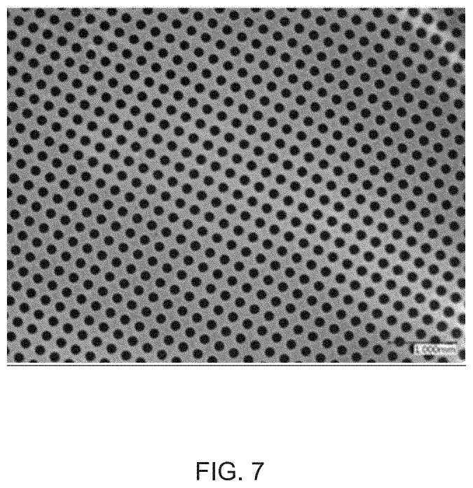

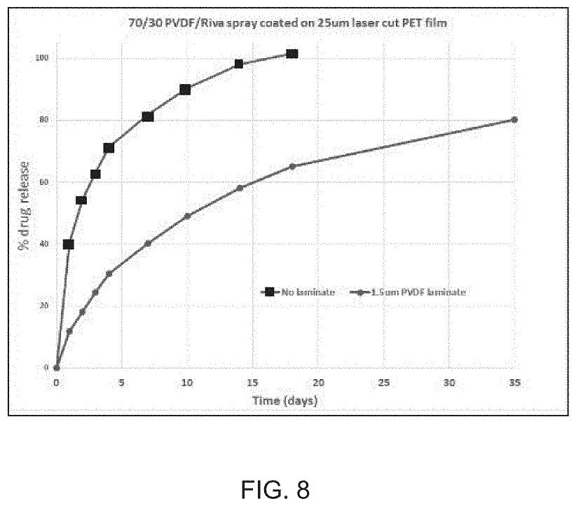

A 25 μm thick PET film was laser cut with 150 μm holes spaced 100 μm apart, as shown in FIG. 7. A solution of PVDF/rivaroxaban (70/30 (wt/wt), 4% total solids in 48/52 (wt/wt) acetone/DMF) was spray coated on the PET film to a coating drug dose density of 6.3 μg drug/mm². One sample of the coated film was over-laminated with a 1.5 μm thick film of PVDF to act as a drug release barrier layer to slow down drug release. Drug release was determined after incubation in PBS/tween 20 at 37° C. for various time points. See FIG. 8. Without the laminate barrier layer, all the drug releases in about two weeks. Adding the laminate barrier layer increases the duration of drug release to well over one month.

### Example 3: Coating Disposed Directly on Device by Spray Coating

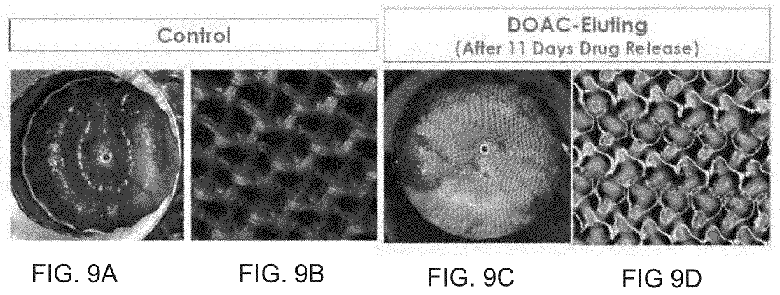

A drug/polymer solution of PVDF-HFP and DOAC was prepared in 80/20 acetone/DMSO (wt/wt). The PVDF-HFP to drug ratio was 60/40 (wt/wt) and the solution solids was 2%. The DOAC drug evaluated was rivaroxaban (Xarelto®). A 24 mm diameter Watchman device was spray-coated with the polymer/drug solution at a flow rate of 10 ml/hr to apply a basecoat. The device was dried for 30 min at 125° C. in a convection oven and then spray-coated with a topcoat solution (2% PVDF-HFP in 100% acetone), at a flow rate of 10 ml/hr. The total basecoat weight was 38.8 mg and the topcoat weight was 18.7 mg. The spray-coated device was then placed in PBS/Tween solution at 37° C. for 11 days to simulate in vivo drug elution. The device was then rinsed with DI water and dried. A PVDF-HFP-only coated control device was also subjected to the same incubation and rinsing protocol. The DOAC-eluting device and the control device were placed in the same container of bovine blood (ACT=210) on an orbital shaker incubator at 37° C. for 15 minutes to assess the thrombogenicity of the two devices. FIG. 9A shows the top of the control device and FIG. 9B is a closeup of the device, showing significant thrombus formation. FIGS. 9C and 9D show the top of the DOAC-eluting device and closeup, where the coating inhibited thrombus formation on the proximal face of the device to a greater degree than did the control device.

### Example 4: Coating Disposed Directly on Masked Device by Spray Coating

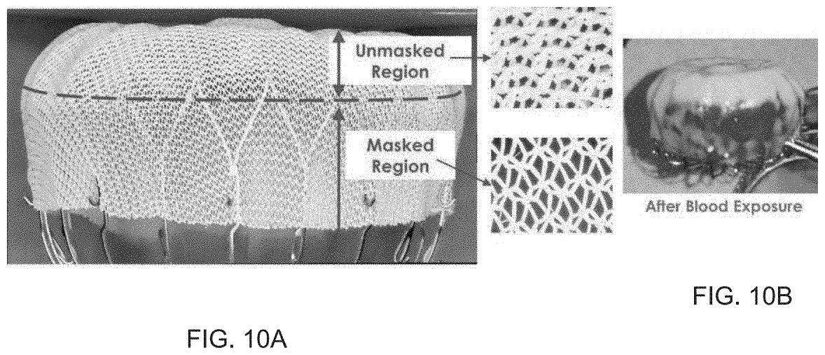

A solution of PVDF-HFP and DOAC was prepared in 40/60 acetone/DMF (wt/wt). The PVDF-HFP to drug ratio was 70/30 (wt/wt) and the solution solids was 4%. The DOAC drug evaluated was apixaban. A 24 mm diameter Watchman device was masked on the back of the device and on the outside of the device using Teflon tape prior to spray-coating, so that only the proximal face of the device would be coated (unmasked region) with the drug/polymer coating. See FIG. 10A. The masked device was then spray-coated with the polymer/drug solution at a flow rate of 20 ml/hr to apply a basecoat. The closeup of the unmasked region shows the coating on the device, as compared to the masked region which is devoid of the coating. The device was dried for 30 min at 125° C. in a convection oven and then spray-coated with a topcoat solution (2% PVDF-HFP in 100% acetone), at a flow rate of 10 ml/hr. The total basecoat weight was 26.6 mg and the topcoat weight was 10.5 mg. The spray-coated device was then placed in PBS/Tween solution at 37° C. for 7 days to simulate in vivo drug elution. The device was then rinsed with DI water and dried. The DOAC-eluting device was placed in bovine blood (ACT=210) on an orbital shaker incubator at 37° C. for 15 minutes to assess the thrombogenicity of the partially coated device. As seen in FIG. 10B, the DOAC-eluting device inhibited thrombus formation only on the proximal face of the device, where the drug/polymer coating was applied, while the distal part of the device shows thrombus formation.

Providing the DOAC directly on the LAA implant provides a localized thrombus prevention desired after implantation of the occlusion device, without the need for systemic oral anticoagulant therapy.

The above embodiments are shown and described as being inserted into the left atrial appendage, however it will be understood that the devices and methods are also useable on the right atrial appendage.

In some embodiments, the plurality of struts 111 of the support structure 110 and/or the plurality of anchors 150 may be formed of or include a metallic material, a metallic alloy, a ceramic material, a rigid or high-performance polymer, a metallic-polymer composite, combinations thereof, and the like. Some examples of some suitable materials may include metallic materials and/or alloys such as stainless steel (e.g., 303, 304v, or 316L stainless steel), nickel-titanium alloy (e.g., nitinol, such as super elastic or linear elastic nitinol), nickel-chromium alloy, nickel-chromium-iron alloy, cobalt alloy, nickel, titanium, platinum, or alternatively, a polymer material, such as a high performance polymer, or other suitable materials, and the like. The word nitinol was coined by a group of researchers at the United States Naval Ordinance Laboratory (NOL) who were the first to observe the shape memory behavior of this material. The word nitinol is an acronym including the chemical symbol for nickel (Ni), the chemical symbol for titanium (Ti), and an acronym identifying the Naval Ordinance Laboratory (NOL).

In some embodiments, the plurality of struts 111 of the support structure 110 and/or the plurality of anchors 150 may be mixed with, may be doped with, may be coated with, or may otherwise include a radiopaque material. Radiopaque materials are understood to be materials capable of producing a relatively bright image on a fluoroscopy screen or another imaging technique such as X-ray during a medical procedure. This relatively bright image aids the user of device in determining its location. Suitable radiopaque materials may include, but are not limited to, bismuth subcarbonate, iodine, gold, platinum, palladium, tantalum, tungsten or tungsten alloy, and the like.

In some embodiments, the membrane 130 may be formed of or include a polymeric material, a metallic or metallic alloy material, a metallic-polymer composite, combinations thereof, and the like. In some embodiments, the membrane 130 is preferably formed of polyethylene terephthalate (PET) such as DACRON®, or expanded polytetrafluoroethylene (ePTFE). Other examples of suitable polymers may include polyurethane, a polyether-ester such as ARNITEL® available from DSM Engineering Plastics, a polyester such as HYTREL® available from DuPont, a linear low density polyethylene such as REXELL®, a polyamide such as DURETHAN® available from Bayer or CRISTAMID® available from Elf Atochem, an elastomeric polyamide, a block polyamide/ether, a polyether block amide such as PEBA available under the trade name PEBAX®, silicones, polyethylene, Marlex high-density polyethylene, polytetrafluoroethylene (PTFE), polyetheretherketone (PEEK), polyimide (PI), and polyetherimide (PEI), a liquid crystal polymer (LCP) alone or blended with other materials.

It should be understood that although the above discussion was focused on a medical device and methods of use within the vascular system of a patient, other embodiments of medical devices or methods in accordance with the disclosure can be adapted and configured for use in other parts of the anatomy of a patient. For example, devices and methods in accordance with the disclosure can be adapted for use in the digestive or gastrointestinal tract. Similarly, the apparatus and/or medical devices described herein with respect to percutaneous deployment may be used in other types of surgical procedures as appropriate. For example, in some embodiments, the medical devices may be deployed in a non-percutaneous procedure, such as an open heart procedure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A device for permanent placement across an atrial appendage ostium in a patient, comprising:
a support structure having a contracted delivery configuration and an expanded deployed configuration defining a radially enlarged portion to permanently engage an interior wall of the atrial appendage;
a membrane attached to the support structure and configured to extend across the ostium of the atrial appendage when the support structure is in the expanded deployed configuration; and
a polymer coating disposed on at least one of the support structure and the membrane, the polymer coating including a direct oral anticoagulant (DOAC) dispersed in a polymer.

2. The device of claim 1, wherein the DOAC is apixaban, rivaroxaban, or edoxaban.

3. The device of claim 1, wherein the polymer coating is disposed on the membrane.

4. The device of claim 3, wherein the DOAC is present in the polymer coating in a ratio of 60/40 to 90/10 weight/weight of polymer to DOAC.

5. The device of claim 3, wherein the DOAC is present in the polymer coating in an amount of between 10-10,000 μg.

6. The device of claim 3, wherein the polymer coating includes the DOAC in a coat density of 100-50,000 ng DOAC/mm² of membrane surface area.

7. The device of claim 3, wherein the polymer is poly(vinylidene fluoride)-co-hexafluoropropylene and the polymer coating has a thickness of about 10-20 μm.

8. The device of claim 1, wherein the polymer coating is disposed directly on the support structure.

9. The device of claim 8, wherein the polymer coating has a thickness of 10 μm.

10. The device of claim 8, wherein the DOAC is present in an amount of 100-300 μg.

11. The device of claim 8, wherein the polymer coating is disposed on a proximal end of the support structure.

12. The device of claim 1, wherein the polymer coating is a 1-10 μm thick film laminated to the membrane.

13. The device of claim 12, wherein the film contains 100-450 μg of the DOAC.

14. The device of claim 12, wherein the film includes a plurality of pores.

15. The device of claim 14, wherein the plurality of pores is 20-150 μm.

16. The device of claim 12, wherein the film is disposed on an atrial face of the membrane.

17. The device of claim 12, wherein the film includes a base layer with the DOAC and a top layer with a modulating compound.

18. A device for permanent placement across a left atrial appendage ostium in a patient, comprising:
a self-expanding support structure having a first contracted shape for delivery and a second expanded shape configured to engage an interior wall of the left atrial appendage, the support structure including a plurality of struts defining an atrial face extending across the left atrial appendage ostium when in the second expanded shape;
a membrane disposed on the atrial face and extending along at least a portion of a side surface of the support structure and configured to extend across the atrial appendage ostium in the second expanded shape; and
a polymer drug coating disposed on one or both of the support structure and the membrane, the polymer drug coating including a direct oral anticoagulant (DOAC) dispersed in a polymer.

19. The device of claim 18, wherein the polymer drug coating is a 1-10 μm thick film laminated directly onto the membrane.

20. A method of making an expandable device for permanent placement across a left atrial appendage ostium in a patient, comprising:
forming an expandable support structure having a contracted delivery configuration and an expanded deployed configuration defining a radially enlarged portion sized to permanently engage an interior wall of the left atrial appendage;
attaching a membrane over at least a proximal end of the support structure; and
applying a polymer coating containing a direct oral anticoagulant dispersed in a polymer to at least one of the support structure and the membrane.
